(19) United States
(12) Patent Application Publication
(10) Pub. No.: US 2026/0315537 A1
(43) Pub. Date: Oct. 15, 2026

(54) COMPOSITIONS AND METHODS FOR CAR-T CELL THERAPY

(71) Applicant: Zahav Bioscience LLC, Philadelphia, PA (US)

(72) Inventors: Giulio Franco PACIOTTI, Phoenix, MD (US); Melik Ziya TURKER, Princeton, NJ (US); Jacob ERSTLING, Philadelphia, PA (US); Nyssa EMERSON, Lambertville, NJ (US); Kevin KIM, Cherry Hill, NJ (US); Philip Francis PASTOR, Glenolden, PA (US); Jennifer A. FRICK, Millville, NJ (US); Linda Marie CHERRY, Doylestown, PA (US); Richard Benjamin RIVERA, Waxhaw, NC (US); Seth SHEFFLER-COLLINS, Philadelphia, PA (US)

(73) Assignee: Zahav Bioscience LLC, Philadelphia, PA (US)

(21) Appl. No.: 19/644,771

(22) Filed: Apr. 10, 2026

Related U.S. Application Data
(60) Provisional application No. 63/787,036, filed on Apr. 11, 2025.

(51) Int. Cl. A61K 35/17; A61K 9/00; A61K 9/51; A61K 33/242; A61K 38/19; A61K 38/20; A61K 38/21; A61K 40/11; A61K 40/31; A61K 40/32; A61K 40/42; A61P 35/00

(52) U.S. Cl. CPC A61K 35/17; A61K 9/0019; A61K 9/51; A61K 33/242; A61K 38/191; A61K 38/208; A61K 38/217; A61K 40/11; A61K 40/31; A61K 40/32; A61K 40/4204; A61K 40/4215; A61P 35/00; A61K 2239/54; A61K 2239/59

(57) ABSTRACT

The present disclosure includes compositions and methods of treating cancerous growth. For example, methods of the present disclosure may include administering a first nanoparticle composition to a subject having the cancerous growth, administering a second nanoparticle composition to the subject having the cancerous growth, and/or administering CAR-T cells to the subject having the cancerous growth. The first nanoparticle composition, the second nanoparticle composition, or both, may include tumor necrosis factor alpha and/or interferon gamma.

### CROSS-REFERENCE TO RELATED APPLICATION

This application claims priority to U.S. Provisional Patent Application No. 63/787,036, filed on Apr. 11, 2025, which is hereby incorporated by reference in its entirety.

### FIELD OF DISCLOSURE

The present disclosure related to compositions and methods for treating cancerous growth. For example, methods of the present disclosure may comprise administering one or more compositions in coordination with chimeric antigen receptor T cells (CAR-T cells).

### INTRODUCTION

Chimeric antigen receptor T cells (CAR-T cells) represent a transformative treatment for cancers, in which a patient's own immune T cells are reprogrammed to attack their cancer. CAR-T cells have been effective against liquid cancers (e.g., occurring in the blood), but have shown limited effectiveness against solid tumors. Too often, the CAR-T cells are unable to reach their intended target.

Therefore, there exists a need for improved methods of administering CAR-T cells for the treatment of cancerous growth.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a method of treating a cancerous growth includes administering a nanoparticle composition to a subject having the cancerous growth. The method may further include administering CAR-T cells to the subject.

According to an aspect of the present disclosure, a method of treating a cancerous growth includes administering a first nanoparticle composition to a subject having the cancerous growth. The method may further include administering CAR-T cells to the subject, about 30 minutes to 8 hours after the first nanoparticle composition is administered. The method may further include administering a second nanoparticle composition to the subject, after administering CAR-T cells to the subject.

### BRIEF DESCRIPTION OF THE DRAWINGS

The accompanying drawings, which are incorporated in and constitute a part of this specification, illustrate various exemplary embodiments, and together with the description, serve to explain the principles of the disclosed embodiments. Any features of an embodiment or example described herein (e.g., composition, formulation, method, etc.) may be combined with any other embodiment or example, and all such combinations are encompassed by the present disclosure. Moreover, the described systems and methods are neither limited to any single aspect nor embodiment thereof, nor to any combinations or permutations of such aspects and embodiments. For the sake of brevity, certain permutations and combinations are not discussed and/or illustrated separately herein.

FIG. 1 is a plot of relative body weights of subjects inoculated with an AsPC-1 tumor line, according to aspects of the present disclosure;

FIG. 2 is a plot of mean tumor volume of subjects inoculated with an AsPC-1 tumor line, according to aspects of the present disclosure;

FIG. 3 is a plot of mean tumor volume of subjects inoculated with an AsPC-1 tumor line, according to aspects of the present disclosure;

FIG. 4 is a plot of mean tumor volume of subjects inoculated with an AsPC-1 tumor line, according to aspects of the present disclosure;

FIG. 5 includes immunohistochemistry images of tumors recovered from subjects inoculated with an AsPC-1 tumor line, according to aspects of the present disclosure;

FIG. 6 is a plot of relative body weights of subjects inoculated with a SKOV3 tumor line, according to aspects of the present disclosure;

FIG. 7 is a plot of mean tumor volume of subjects inoculated with an SKOV3 tumor line, according to aspects of the present disclosure;

FIG. 8 is a plot of mean tumor volume of subjects inoculated with an SKOV3 tumor line, according to aspects of the present disclosure;

FIG. 9 is a plot of mean tumor volume of subjects inoculated with an SKOV3 tumor line, according to aspects of the present disclosure;

FIG. 10 is a plot of mean tumor volume of subjects inoculated with an SKOV3 tumor line, according to aspects of the present disclosure;

FIG. 11 is a plot of mean tumor volume of subjects inoculated with an SKOV3 tumor line, according to aspects of the present disclosure; and

FIGS. 12A-G include flow cytometry plots from a tumor sample extracted from a subject inoculated with an SKOV3 tumor line, according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

Unless defined otherwise, all technical and scientific terms used herein have the same meaning as is commonly understood by one of ordinary skill in the art to which this disclosure belongs. Although any suitable methods and materials (e.g., similar or equivalent to those described herein) can be used in the practice or testing of the present disclosure, particular exemplary methods are now described.

As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” For the terms “for example” and “such as,” and grammatical equivalences thereof, the phrase “and without limitation” is understood to follow unless explicitly stated otherwise.

As used herein, the terms “about” or “approximately” are meant to account for variations due to experimental error. When applied to numeric values, the terms “about” or “approximately” may indicate a variation of +/−5% from the disclosed numeric value, unless a different variation is specified. When applied to pH values, the terms “about” or “approximately” may indicate a variation of +/−0.05. As used herein, the singular forms “a,” “an,” and “the” include plural referents unless the context clearly dictates otherwise. Further, all ranges are understood to be inclusive of endpoints, e.g., from 1 microgram (μg) to 5 μg would include masses of 1 μg, 5 μg, and all masses between 1 μg and 5 μg.

The present disclosure includes compositions and methods for conditioning the tumor microenvironment to enhance penetration of therapeutics and targeting of therapeutics to the cancerous growth. For example, compositions described herein may include a nanoparticle composition configured to improve the activity of therapeutic CAR-T cells. Compositions described herein may be used in combination with CAR-T cells to target cancer types that have been traditionally resistant to CAR-T cell therapy (e.g., solid tumors, tumors with relatively low vascularity, and tumors with relatively high interstitial fluid pressure (IFP)).

CAR-T cells represent a form of immunotherapy in which a patient's own immune T cells are reprogrammed to recognize and attack cancer cells. CAR-T cell therapy has demonstrated efficacy in treating hematologic malignancies, such as liquid cancers occurring in the blood. However, CAR-T cell therapy has shown limited effectiveness against solid tumors.

Solid tumors may present challenges for CAR-T cell therapy due to limited infiltration of effector T cells across the tumor vasculature and into the tumor microenvironment. In some cases, angiogenesis associated with tumor growth may lead to downregulation of adhesion molecules, such as intercellular adhesion molecule-1 (ICAM-1), on tumor vascular endothelial cells. The downregulation of adhesion molecules may reduce the ability of CAR-T cells to adhere to and transmigrate through the tumor vasculature. Additionally, solid tumors may exhibit elevated interstitial fluid pressure, which may further limit penetration of systemically delivered therapeutics, including CAR-T cells.

The interstitial fluid pressure may limit penetration of systemically delivered cancer therapies to solid tumors. For example, tumors with relatively high interstitial fluid pressure may be resistant to traditional CAR-T cell therapies. Without being bound by theory, the compositions and methods described herein may work to selectively disrupt tumor vasculature and/or increase permeability of tumor blood vessels. Disruption of the tumor vasculature may be detected by measuring a loss of endothelial cell marker CD34 and/or endothelial cell death. Disruption of the tumor vasculature may cause a reduction in tumor interstitial fluid pressure. The compositions and methods described herein may cause or assist disruptions and/or degradation of tumor vasculature, reducing interstitial fluid pressure, thereby improving intra-tumoral accumulation of therapeutics (e.g., CAR-T cells).

In some aspects, the nanoparticle composition may selectively disrupt tumor vasculature and increase permeability of tumor blood vessels. Disruption of the tumor vasculature may cause a reduction in tumor interstitial fluid pressure, thereby improving intra-tumoral accumulation of CAR-T cells. In some cases, the nanoparticle composition may induce expression of adhesion molecules, such as E-selectin and ICAM-1, on tumor vascular endothelial cells. The expression of adhesion molecules may facilitate leukocyte rolling, adhesion, and transmigration through the endothelial barrier.

In some aspects, the nanoparticle composition may induce secretion of chemokines within the tumor microenvironment. The chemokines may establish a chemokine gradient that directs CAR-T cells toward antigen expressing tumor cells. In some cases, the nanoparticle composition may enhance major histocompatibility complex class I (MHC-I) expression on tumor cells, which may further promote immune recognition and cytotoxic activity against the tumor cells.

The compositions and methods described herein may be used to treat cancer types that have been traditionally resistant to CAR-T cell therapy, including solid tumors, tumors with relatively low vascularity, and/or tumors with relatively high interstitial fluid pressure. Sequential or parallel administration of the nanoparticle compositions with CAR-T cells may improve infiltration of CAR-T cells into solid tumors and/or improve activity of CAR-T cells in solid tumors.

The compositions described herein may include a colloidal metal nanoparticle. The colloidal metal nanoparticles may be bound to one or more secondary moieties (e.g., secondary molecules), such as targeting, biocompatibility (e.g., shielding), and/or therapeutic moeities.

The colloid metal nanoparticle may include a colloidal gold nanoparticle, such as those disclosed in U.S. Patent Application Publication No. 2025/0034221 A1; U.S. Pat. Nos. 7,229,841; 7,387,900; 8,785,202; RE42524; 7,960,145; 8,486,663; 5,587,294; 5,965,379; 6,274,552; 8,137,989; 6,407,218; 6,528,051; 7,790,167; 7,951,614; 8,435,801; U.S. Patent Application No. 2007/0160572; U.S. Patent Application Publication No. 2007/0231408; U.S. Patent Application Publication No. 2012/0134956; U.S. Patent Application Publication No. 2008/0033385; U.S. Patent Application Publication No. 2008/0215030; U.S. Patent Application Publication No. 2009/00035265; U.S. Patent Application Publication No. 2001/0055581; U.S. Patent Application Publication No. 2005/0175584; U.S. Patent Application Publication No. 2005/0175583; U.S. Patent Application Publication No. 2009/0104114; U.S. Patent Application Publication No. 2011/0014118; and U.S. Patent Application Publication No. 2015/0018486; the entire contents of each of which are incorporated by reference herein.

In some aspects, the nanoparticle may comprise a biocompatible core, such as, for example, silica, or a biocompatible polymer (e.g., poly(lactic-co-glycolic acid) (PLGA)).

The one or more therapeutic moieties may include tumor necrosis factor (TNF), tumor necrosis factor-alpha (TNFα), endostatin, angiostatin, thalidomide, taxol, melphalan, paclitaxel, vinblastine, vincristine, doxorubicin, acyclovir, cisplatin, tacrine, 5-fluorouracil, mitaxantrone, VM-16, etoposide, VM-26, teniposide, taxanes, interleukin-1 (IL-1), interleukin-2 (IL-2), interleukin-3 (IL-3), interleukin-4 (IL-4), interleukin-5 (IL-5), interleukin-6 (IL-6), interleukin-7 (IL-7), interleukin-8 (IL-8), interleukin-9 (IL-9), interleukin-10 (IL-10), interleukin-11 (IL-11), interleukin-12 (IL-12), interleukin-13 (IL-13), lipid A, phospholipase A2, endotoxins, staphylococcal enterotoxin B, type I interferon, type II interferon, interferon-gamma (IFN7), transforming growth factor (TGF), transforming growth factor-alpha (TGFα), transforming growth factor-beta (TGFβ), lymphotoxin, migration inhibition factor, granulocyte-macrophage colony stimulating factor, monocyte-macrophage colony stimulating factor, granulocyte colony-stimulating factor, angiogenin, fibroblast growth factor, cancer cell specific antigens, MART, MAGE, BAGE, B7 family molecules and receptors, CD40 ligand, CD40 receptor, basic fibroblast growth factor, macrophage inflammatory protein, PD1/PD-L1 agonists, PD1/PD-L1 antagonists, and vascular endothelial growth factor (VEGF).

The one or more therapeutic moieties may include one or more chemotherapeutic agents. The one or more chemotherapeutic agents may include taxol, melphalan, paclitaxel, vinblastine, vincristine, doxorubicin, acyclovir, cisplatin, tacrine, 5-fluorouracil, mitaxantrone, VM-16, etoposide, VM-26, teniposide, taxanes, or combinations thereof.

The one or more therapeutic moieties may include one or more immunostimulatory agents. The one or more immunostimulatory agents may include lipid A, phospholipase A2, endotoxins, staphylococcal enterotoxin B, type I interferon, type II interferon, lymphotoxin, migration inhibition factor, macrophage inflammatory protein, or combinations thereof.

The one or more therapeutic moieties may include one or more growth factors. The one or more growth factors may include transforming growth factor (TGF), transforming growth factor-alpha (TGFα), transforming growth factor-beta (TGFβ), granulocyte-macrophage colony stimulating factor, monocyte-macrophage colony stimulating factor, granulocyte colony-stimulating factor, fibroblast growth factor, basic fibroblast growth factor, or combinations thereof.

The one or more therapeutic moieties may include one or more cancer antigens. The one or more cancer antigens may include cancer cell specific antigens, MART, MAGE, BAGE, or combinations thereof.

The one or more therapeutic moieties may include one or more immune checkpoint modulators. The one or more immune checkpoint modulators may include B7 family molecules and receptors, CD40 ligand, CD40 receptor, PD1/PD-L1 agonists, PD1/PD-L1 antagonists, or combinations thereof.

The one or more therapeutic moieties may include one or more angiogenic factors or anti-angiogenic agents. The one or more angiogenic factors may include vascular endothelial growth factor (VEGF) or angiogenin. The one or more anti-angiogenic agents may include endostatin, angiostatin, thalidomide, or combinations thereof.

In some aspects, the one or more therapeutic moieties may include IFNγ. IFNγ may modulate PD1/PD-L1 inhibition of CAR-T cell activity on tumor cells. In some cases, administration of nanoparticle compositions comprising IFN7 may induce intercellular adhesion molecule-1 (ICAM-1) expression.

The one or more targeting moieties may include targeting peptides configured to localize the nanoparticle composition to tumor vasculature. In some aspects, the targeting peptides may include cyclic RGDfK peptides, NGR peptides, neuropilin-binding sequences, or combinations thereof. In some cases, the one or more targeting moieties may include tumor targeting ligands such as epidermal growth factor receptor (EGFR), human epidermal growth factor receptor 2 (HER2), mesothelin, claudin 18.2 (CLDN18.2), c-Met, EpCAM, ephrin type-A receptor 2 (EphA2), folate receptor alpha (FR-alpha), disialoganglioside GD2 (GD2), glypican-3 (GPC-3), mucin 1 (MUC-1), programmed death-ligand 1 (PD-L1), programmed cell death protein 1 (PD-1), prostate stem cell antigen (PSCA), prostate-specific membrane antigen (PSMA), receptor tyrosine kinase-like orphan receptor 1 (ROR1), uroplakin 1A (UPK1A), vascular endothelial growth factor receptor 2 (VEGFR2), or a combination thereof.

In some aspects, the one or more therapeutic moieties may include IFNγ. IFNγ may modulate PD1/PD-L1 inhibition of CAR-T cell activity on tumor cells. For example, administration of compositions of the present disclosure may induce ICAM-1 expression.

One or more biocompatibility moieties may include polyethylene glycol (PEG) or derivatives thereof. For example, the one or more biocompatibility agents may include PEG, polyethylene glycol-thiol (PEG-thiol), or a derivative thereof. In some aspects, the one or more biocompatibility agents may include PEG, PEG-thiol, and/or PEG derivatives, where the PEG, PEG-thiol, and/or PEG derivatives have different sizes. The biocompatibility moieties may reduce uptake and clearance of the nanoparticle composition by portions of the innate immune system, such as the Monocyte Phagocytic System (MPS).

The compositions described herein may include one or more excipients. The one or more excipients may include pharmaceutically acceptable excipients such as fructose, maltose, galactose, glucose, D-mannose, sorbose, lactose, sucrose, trehalose, cellobiose, raffinose, melezitose, maltodextrin, dextran, starch, mannitol, xylitol, maltitol, lactitol, xylitol sorbitol (blucitol), myoinositol, or a combination thereof. For example, the one or more excipients may include mannitol, sucrose, or a combination of mannitol and sucrose.

In some cases, the one or more excipients may function as stabilizers, bulking agents, or lyoprotectants for the nanoparticle composition. The one or more excipients may maintain the stability of the nanoparticle composition (e.g., stability of a colloidal metal within the composition) during storage and administration.

In some aspects, the one or more therapeutic moieties may include TNFα. TNFα may act as a vascular disrupting agent, selectively destroying tumor vasculature, reducing tumor interstitial fluid pressure, increase the uptake of follow-on chemotherapy, and/or recruit immune system cells to the site of the tumor. Collectively, these effects may result in significant anti-tumor responses. In some aspects, the one or more therapeutic moieties may include TNFα and IFNγ.

A nanoparticle composition may comprise both tumor necrosis factor alpha (TNFα) and interferon gamma (IFNγ) conjugated to the nanoparticle core (e.g., a colloidal gold core). A nanoparticle composition comprising both TNFα and IFNγ bound to a single nanoparticle surface may be referred to as a dual cytokine nanoparticle composition. The dual cytokine nanoparticle composition may include a colloidal gold nanoparticle or other biocompatible core, such as silica or poly(lactic-co-glycolic acid) (PLGA). In some cases, both TNFα and IFN7 may bind directly to the surface of the colloidal gold nanoparticle.

The dual cytokine nanoparticle composition may have a molar ratio of TNFα to IFNγ configured to achieve a desired biological effect. In some aspects, the dual cytokine nanoparticle composition may have a high TNFα to low IFNγ molar ratio. A dual cytokine nanoparticle composition having a high TNFα to low IFNγ molar ratio may induce expression of adhesion molecules on tumor vascular endothelial cells. The adhesion molecules may include E-selectin, intercellular adhesion molecule-1 (ICAM-1), vascular cell adhesion molecule-1 (VCAM-1), or combinations thereof. E-selectin may slow the movement of CAR-T cells over the tumor blood vessel wall through a bind and release mechanism with sialyl Lewisx (sLex) carbohydrates. ICAM-1 may bind to the lymphocyte function-associated antigen (LFA) receptor on CAR-T cells, causing firm adhesion of the CAR-T cells to endothelial cells and regulating transmigration of the CAR-T cells through tumor blood vessels.

A high TNFα to low IFNγ molar ratio may refer to a TNFα to IFN7 ratio of approximately 50:1 to approximately 10:1, such as, for example, approximately 40:1 to approximately 20:1, approximately 50:1, approximately 40:1, approximately 30:1; approximately 20:1, or approximately 10:1.

In some aspects, the dual cytokine nanoparticle composition may have a low TNFα to high IFN7 molar ratio. A dual cytokine nanoparticle composition having a low TNFα to high IFNγ molar ratio may establish a chemokine gradient within the tumor microenvironment. The chemokine gradient may include CXCL9, CXCL10, CXCL11, or combinations thereof. The chemokine gradient may direct CAR-T cells, natural killer (NK) cells, and T cells toward cancer cells expressing cognate antigens. In some cases, the dual cytokine nanoparticle composition having a low TNFα to high IFNγ molar ratio may stimulate tumor cells and stromal fibroblasts to secrete chemokines.

A low TNFα to high IFNγ molar ratio may refer to a TNFα to IFNγ ratio of approximately 1:50 to approximately 1:10, such as, for example, approximately 1:40 to approximately 1:20, approximately 1:50, approximately 1:40, approximately 1:30; approximately 1:20, or approximately 1:10.

In some aspects, a single dual cytokine nanoparticle composition comprising both TNFα and IFNγ may be capable of inducing leukocyte adherence, transmigration through the tumor vascular endothelium, and chemokine homing of leukocytes to tumors. The dual cytokine nanoparticle composition may increase pharmacokinetic exposure to both TNFα and IFNγ. In some cases, the dual cytokine nanoparticle composition may induce expression of major histocompatibility complex (MHC) class I molecules or human leukocyte antigen (HLA) molecules on tumor cells. The induction of MHC-I or HLA expression may promote immune recognition and cytotoxic activity against tumor cells.

The nanoparticle composition may selectively disrupt tumor vasculature while leaving normal blood vessels unaffected. In some aspects, the nanoparticle composition may selectively destroy tumor vasculature and/or increase permeability of tumor blood vessels. Disruption of the tumor vasculature may be detected by measuring a loss of endothelial cell marker CD34 and/or endothelial cell death. In some cases, vascular disruption may be determined by quantitative immunohistochemistry for vascular endothelial cell markers such as CD31, CD34, or von Willebrand Factor, pericyte markers such as PDGFR-β, or basement membrane markers such as collagen or laminin.

The nanoparticle composition may decrease tumor interstitial fluid pressure. Disruption of the tumor vasculature may cause a reduction in tumor interstitial fluid pressure. In some aspects, the reduction in tumor interstitial fluid pressure may improve intra-tumoral accumulation of therapeutics, including CAR-T cells. The nanoparticle composition may eliminate or reduce the vascular barrier, thereby increasing CAR-T cell uptake into the tumor microenvironment.

In some aspects, the nanoparticle composition may induce expression of adhesion molecules on tumor vascular endothelial cells. The adhesion molecules may include intercellular adhesion molecule-1 (ICAM-1), E-selectin, vascular cell adhesion molecule-1 (VCAM-1), or combinations thereof. In some cases, the nanoparticle composition may interact with the tumor endothelium, engaging tumor necrosis factor receptor 1 (TNFR1) and interferon gamma receptor (IFNγR1/2) signaling pathways to induce E-selectin, ICAM-1, and VCAM-1 expression.

E-selectin may slow the movement of CAR-T cells over the tumor blood vessel wall through a bind and release mechanism with sialyl Lewisx (sLex) carbohydrates. ICAM-1 may bind to the lymphocyte function-associated antigen (LFA) receptor on CAR-T cells. The binding of ICAM-1 to the LFA receptor may cause firm adhesion of CAR-T cells to endothelial cells and may regulate transmigration of CAR-T cells through tumor blood vessels. The adhesion molecules may facilitate leukocyte rolling, adhesion, and transmigration through the endothelial barrier.

In some aspects, the nanoparticle composition comprising the vascular disrupting agent may enhance the uptake of secondary therapeutics administered after the nanoparticle composition. The secondary therapeutics may include CAR-T cells, chemotherapeutic agents, or combinations thereof. In some cases, the nanoparticle composition may increase the number of CAR-T cells and CD8+ T cells that cross through the tumor vasculature.

Sequential or parallel administration of the nanoparticle compositions described herein with CAR-T cells may improve or enhance activity of CAR-T cells in solid tumors. Further described herein are methods of using compositions (e.g., including nanomedicine) to treat cancer and methods of using the compositions to enhance CAR-T cell activity.

The methods described herein may include administering CAR-T cells to a subject having a cancerous growth. In some aspects, CAR-T cells may be derived from a patient's own T cells, which are collected, genetically modified to express the chimeric antigen receptor, expanded in culture, and administered back to the patient.

A chimeric antigen receptor may include an extracellular antigen-binding domain, a transmembrane domain, and one or more intracellular signaling domains. The extracellular antigen-binding domain may be configured to recognize and bind to a tumor-associated antigen. The transmembrane domain may anchor the chimeric antigen receptor to the T cell membrane. The one or more intracellular signaling domains may include costimulatory domains and activation domains configured to trigger T cell activation upon antigen binding.

In some aspects, the one or more intracellular signaling domains may include a costimulatory domain derived from 4-1BB (CD137). The 4-1BB costimulatory domain may enhance T cell proliferation, survival, and persistence. In some cases, the one or more intracellular signaling domains may include a CD3ζ (CD3 zeta) activation domain. The CD3ζ activation domain may provide the primary activation signal for T cell cytotoxic activity.

The CAR-T cells may include MSLN-2-TM-41BB-CD3ζ CAR-T cells. MSLN-2-TM-41BB-CD3ζ CAR-T cells may include a chimeric antigen receptor comprising an extracellular domain configured to bind mesothelin, a transmembrane domain, a 4-1BB costimulatory domain, and a CD3ζ activation domain. Mesothelin is a tumor-associated antigen that may be expressed on the surface of cancer cells in various tumor types. In some aspects, MSLN-2-TM-41BB-CD3ζ CAR-T cells may be administered to a subject having pancreatic cancer. Pancreatic cancer cells may express mesothelin, and MSLN-2-TM-41BB-CD3ζ CAR-T cells may be configured to recognize and target mesothelin-expressing pancreatic cancer cells.

The CAR-T cells may include HER2-TM-41BB-CD3ζ CAR-T cells. HER2-TM-41BB-CD3ζ CAR-T cells may include a chimeric antigen receptor comprising an extracellular domain configured to bind human epidermal growth factor receptor 2 (HER2), a transmembrane domain, a 4-1BB costimulatory domain, and a CD3ζ activation domain. HER2 is a tumor-associated antigen that may be overexpressed on the surface of cancer cells in various tumor types. In some aspects, HER2-TM-41BB-CD3ζ CAR-T cells may be administered to a subject having ovarian cancer. Ovarian cancer cells may overexpress HER2, and HER2-TM-41BB-CD3ζ CAR-T cells may be configured to recognize and target HER2-expressing ovarian cancer cells.

The CAR-T cells may include CD19-targeting CAR-T cells. CD19-targeting CAR-T cells may include a chimeric antigen receptor comprising an extracellular domain configured to bind CD19. CD19 is a cell surface marker expressed on B cells and may be expressed on certain cancer cells, such as B cell malignancies. In some aspects, CD19-targeting CAR-T cells may be administered to a subject having a hematologic malignancy or a solid tumor expressing CD19. In some cases, CD19-targeting CAR-T cells may be commercially available CAR-T cells.

In some aspects, the CAR-T cells may target tumor-associated antigens including HER2, mesothelin, EpCAM, MUC1, or combinations thereof. In some cases, the methods described herein may include administering other engineered immune effector cells expressing a chimeric antigen receptor, such as CAR-NK cells, CAR-iNKT cells, or CAR-gamma delta T cells.

The CAR-T cells may be administered in combination with the nanoparticle compositions described herein. In some aspects, administration of the nanoparticle composition prior to administration of the CAR-T cells may enhance penetration of the CAR-T cells into solid tumors. The nanoparticle composition may condition the tumor microenvironment by disrupting tumor vasculature, reducing interstitial fluid pressure, inducing expression of adhesion molecules on tumor vascular endothelial cells, and establishing a chemokine gradient to direct CAR-T cells toward tumor cells. In some cases, the combination of the nanoparticle composition and the CAR-T cells may result in increased intratumoral CAR-T cell infiltration and cytotoxicity relative to CAR-T cell therapy alone.

In some aspects, the CAR-T cells may be administered after the nanoparticle composition is administered. Administration of the nanoparticle composition prior to administration of the CAR-T cells may allow the nanoparticle composition to condition the tumor microenvironment before the CAR-T cells are introduced. In some cases, the nanoparticle composition may disrupt tumor vasculature, reduce tumor interstitial fluid pressure, and induce expression of adhesion molecules on tumor vascular endothelial cells prior to administration of the CAR-T cells. The conditioning of the tumor microenvironment may enhance penetration of the CAR-T cells into solid tumors.

In some aspects, the CAR-T cells may be administered about 15 minutes to about 8 hours after the nanoparticle composition is administered. In some cases, the CAR-T cells may be administered about 30 minutes to about 2 hours after the nanoparticle composition is administered, the CAR-T cells may be administered about 1 hour to about 2 hours after the nanoparticle composition is administered, the CAR-T cells may be administered about 1 hour after the nanoparticle composition is administered, or the CAR-T cells may be administered about 15 minutes to about 45 minutes after the nanoparticle composition is administered.

In some aspects, the timing of administration may be selected based on the desired effect of the nanoparticle composition on the tumor microenvironment. For example, administration of the CAR-T cells about 15 minutes to about 8 hours (e.g., about 1 hour) after administration of the nanoparticle composition may allow sufficient time for the nanoparticle composition to induce vascular disruption and expression of adhesion molecules while maintaining elevated levels of the nanoparticle composition at the tumor site. In some cases, the timing of administration may be adjusted based on the type of cancerous growth, the composition of the nanoparticle composition, or the type of CAR-T cells being administered.

The nanoparticle composition and the CAR-T cells may be administered to a subject according to various timing regimens. In some aspects, the nanoparticle composition and the CAR-T cells may be administered simultaneously. Simultaneous administration may include administering the nanoparticle composition and the CAR-T cells at approximately the same time or within a short interval of each other.

In some aspects, a nanoparticle composition may be administered after administration of CAR-T cells. For example CAR-T cells may be administered and allowed to proliferate within the subject for about 1 day to about 21 days, such as, for example, about 7 day to about 14 days, about 1 day to about 14 days, or about 10 to about 18 days. After the CAR-T cells are allowed to proliferate within the subject, a nanoparticle composition may be administered. Administering the nanoparticle composition after the proliferation of CAR-T cells may provide for increased CAR-T cells populations in the tumor microenvironment while the nanoparticle is administered, increasing infiltration of CAR-T cells into the cancerous cells.

In some aspects, a method of treating a cancerous growth may include administering a first nanoparticle composition to a subject, administering CAR-T cells to the subject about 30 minutes to about 8 hours after the first nanoparticle composition is administered, and administering a second nanoparticle composition to the subject after administering the CAR-T cells. In some cases, the CAR-T cells may be administered about 30 minutes to about 2 hours after the first nanoparticle composition is administered. In some aspects, the CAR-T cells may be administered about 1 hour to about 2 hours after the first nanoparticle composition is administered. The first nanoparticle composition may be identical to the second nanoparticle composition, or the first nanoparticle composition may be different than the second nanoparticle composition.

Compositions of the present disclosure (e.g., the first nanoparticle composition, the second nanoparticle composition, and/or the CAR-T cells) may be administered to a subject via one or more routes of administration. In some aspects, the compositions may be administered intravenously to the subject. Intravenous administration of the compositions may allow the nanoparticle compositions and/or CAR-T cells to circulate through the bloodstream and accumulate at the site of the cancerous growth. In some cases, the nanoparticle compositions and/or CAR-T cells may be administered as an intravenous injection or an intravenous infusion.

In some aspects, the nanoparticle composition may be formulated for intravenous administration. The nanoparticle composition may be suspended or dissolved in a pharmaceutically acceptable carrier, such as an aqueous buffer solution. In some cases, the aqueous buffer solution may comprise sodium phosphate, polyethylene glycol, human serum albumin, or combinations thereof. In some aspects, the CAR-T cells may be suspended in a pharmaceutically acceptable carrier for intravenous administration. The pharmaceutically acceptable carrier for the CAR-T cells may comprise phosphate buffered saline, fetal bovine serum, human serum albumin, or combinations thereof.

In some aspects, the nanoparticle compositions and/or the CAR-T cells may be administered via other parenteral routes of administration. The other parenteral routes of administration may include intramuscular injection, subcutaneous injection, intradermal injection, or a combination thereof. In some cases, the nanoparticle composition may be administered via intratumoral injection to deliver the nanoparticle composition directly to the site of the cancerous growth.

In some aspects, a method of treating a cancerous growth may include administering a first nanoparticle composition and a second nanoparticle composition to a subject having the cancerous growth. The first nanoparticle composition and the second nanoparticle composition may be administered at different times during a treatment regimen. In some cases, the first nanoparticle composition may be administered prior to administration of CAR-T cells, and the second nanoparticle composition may be administered after administration of the CAR-T cells.

In some aspects, the second nanoparticle composition may be administered after the CAR-T cells are administered. Administration of the second nanoparticle composition after CAR-T cell administration may provide additional conditioning of the tumor microenvironment to support CAR-T cell activity. In some cases, the second nanoparticle composition may be administered about 1 day to about 10 days after the CAR-T cells are administered. In some aspects, the second nanoparticle composition may be administered about 5 days after the CAR-T cells are administered. In some cases, the second nanoparticle composition may be administered about 3 days to about 7 days after the CAR-T cells are administered.

In some aspects, a method of treating a cancerous growth may include administering multiple doses of a nanoparticle composition to a subject. The multiple doses may include one, two, three, or more injections of the nanoparticle composition. In some cases, the number of injections may be selected based on the degree of vascular disruption achieved in the tumor microenvironment. In some aspects, dynamic contrast-enhanced magnetic resonance imaging (DCE-MRI) may be used to confirm vascular disruption and to determine the number of nanoparticle composition treatments administered prior to administration of CAR-T cells.

Multiple doses of the nanoparticle composition may be delivered every day, every two days, every three days, every four days, every five days, every week, every two weeks, every three weeks, or monthly.

In some aspects, a treatment regimen may include administering one injection of the nanoparticle composition prior to administration of CAR-T cells. A single injection of the nanoparticle composition may be sufficient to disrupt tumor vasculature and condition the tumor microenvironment for CAR-T cell infiltration. In some cases, a treatment regimen may include administering two injections of the nanoparticle composition. In some aspects, a treatment regimen may include administering three injections of the nanoparticle composition. The two or three injections may be administered on consecutive days or on non-consecutive days prior to administration of CAR-T cells.

In some aspects, a treatment regimen may include administering a first injection of the nanoparticle composition, administering CAR-T cells after the first injection, and administering a second injection of the nanoparticle composition after the CAR-T cells are administered. In some cases, the treatment regimen may include administering a third injection of the nanoparticle composition after the second injection. The multiple injections may maintain vascular disruption and support continued CAR-T cell infiltration into the tumor microenvironment over an extended treatment period.

The nanoparticle composition may be administered at various doses depending on the therapeutic application and the subject being treated. In some aspects, the nanoparticle composition may be administered at a dose of about 50 μg to about 1000 μg of TNFα, such as for example, about 50 μg to about 800 μg, about 50 μg to about 700 μg, about 50 μg to about 600 μg, about 50 μg to about 500 μg, about 50 μg to about 400 μg, or about 50 μg to about 300 μg. For example, the nanoparticle composition may be administered based on a surface area of the subject. For example, the nanoparticle composition may be administered at a dose of about 25 μg/m² to about 500 μg/m² of TNFα, such as for example, about 25 μg/m² to about 400 μg/m2, about 25 μg/m2 to about 350 μg/m2, about 25 μg/m2 to about 300 μg/m2, about 25 μg/m2 to about 250 μg/m2, about 25 μg/m2 to about 200 μg/m2, or about 25 μg/m2 to about 150 μg/m2.

In some aspects, the dose of the nanoparticle composition may be selected based on the type of cancerous growth, the size of the cancerous growth, or the condition of the subject. In some cases, lower doses of the nanoparticle composition may be administered when the nanoparticle composition is administered in combination with CAR-T cells. In some aspects, the dose of the nanoparticle composition may be adjusted based on the observed response of the cancerous growth to treatment.

The CAR-T cells may be administered at various doses depending on the therapeutic application and the subject being treated. In some aspects, the CAR-T cells may be administered at a dose of about 0.1×10⁶ cells to about 10×10⁹ cells, per kilogram of subject. In some aspects, the dose of CAR-T cells may be selected based on the type of cancerous growth, the size of the cancerous growth, or the condition of the subject. In some cases, lower doses of CAR-T cells may be administered when the CAR-T cells are administered in combination with a nanoparticle composition. Administration of the nanoparticle composition may enhance penetration of CAR-T cells into the tumor microenvironment, which may allow for administration of lower doses of CAR-T cells while maintaining therapeutic efficacy. In some aspects, the dose of CAR-T cells may be adjusted based on the observed response of the cancerous growth to treatment.

The methods described herein may be used to treat various types of cancerous growths. In some aspects, the cancerous growth may include a tumor. In some aspects, the cancerous growth may include a solid tumor with elevated interstitial fluid pressure. An elevated interstitial fluid pressure may refer to an interstitial fluid pressure greater than or equal about 2.5 mmHg, such as, for example, greater than or equal to about 5.0 mmHg, greater than or equal to about 7.5 mmHg, greater than or equal to about 10.0 mmHg, greater than or equal to about 15.0 mmHg, greater than or equal to about 20.0 mmHg, greater than or equal to about 25.0 mmHg, greater than or equal to about 27.5 mmHg, or greater than or equal to about 30.0 mmHg, about 2.5 mmHg to about 40.0 mmHg, about 5.0 mmHg to about 40.0 mmHg, about 7.5 mmHg to about 40.0 mmHg, about 10.0 mmHg to about 40.0 mmHg, about 15.0 mmHg to about 40.0 mmHg, about 20.0 mmHg to about 40.0 mmHg, about 22.5 mmHg to about 40.0 mmHg, about 25.0 mmHg to about 40.0 mmHg, about 27.5 mmHg to about 40.0 mmHg, or about 30.0 mmHg to about 40.0 mmHg.

Interstitial fluid pressure may limit penetration of systemically delivered cancer therapies to solid tumors. Tumors with relatively high interstitial fluid pressure may be resistant to traditional CAR-T cell therapies. The nanoparticle compositions described herein may selectively disrupt tumor vasculature and reduce tumor interstitial fluid pressure, thereby improving intra-tumoral accumulation of CAR-T cells.

Methods of the present disclosure may include measuring an interstitial fluid pressure, and based on the measurement of the interstitial fluid pressure, administering one or more nanoparticle compositions and/or CAR-T cells, according to the methods of the present disclosure. For example, based on a determination that the measured interstitial fluid pressure meets a threshold interstitial fluid pressure, a therapy including one or more nanoparticle compositions and/or CAR-T cells may be applied or adjusted. Adjusting a therapy may include adjusting a dosing amount, adjusting a timing of an administration of one or more nanoparticle compositions and/or CAR-T cells, and/or adjusting an amount of administrations of one or more nanoparticle compositions and/or CAR-T cells. The threshold interstitial fluid pressure may be about 2.5 mmHg, such as, for example, about 5.0 mmHg, about 7.5 mmHg, about 10.0 mmHg, about 15.0 mmHg, about 20.0 mmHg, about 25.0 mmHg, about 27.5 mmHg, or about 30.0 mmHg.

In some aspects, the cancerous growth may include a tumor with relatively low vascularity. Tumors with relatively low vascularity may exhibit reduced penetration of systemically delivered therapeutics, including CAR-T cells. The nanoparticle compositions described herein may condition the tumor microenvironment to enhance penetration of CAR-T cells into tumors with relatively low vascularity.

In some aspects, the cancerous growth may include pancreatic cancer. Pancreatic cancer cells may express mesothelin, and CAR-T cells configured to recognize and target mesothelin-expressing pancreatic cancer cells may be administered in combination with the nanoparticle compositions described herein. In some cases, MSLN-2-TM-41BB-CD3ζ CAR-T cells may be administered to a subject having pancreatic cancer.

In some aspects, the cancerous growth may include ovarian cancer. Ovarian cancer cells may overexpress human epidermal growth factor receptor 2 (HER2), and CAR-T cells configured to recognize and target HER2-expressing ovarian cancer cells may be administered in combination with the nanoparticle compositions described herein. In some cases, HER2-TM-41BB-CD3ζ CAR-T cells may be administered to a subject having ovarian cancer.

In some aspects, the cancerous growth may include mantle cell lymphoma (MCL). The nanoparticle compositions described herein may enhance penetration of CAR-T cells into MCL tumors. In some cases, CD19-targeting CAR-T cells may be administered to a subject having mantle cell lymphoma.

In some aspects, the methods described herein may be used to treat cancer types that have been traditionally resistant to CAR-T cell therapy. The nanoparticle compositions may condition the tumor microenvironment by disrupting tumor vasculature, reducing interstitial fluid pressure, inducing expression of adhesion molecules on tumor vascular endothelial cells, and establishing a chemokine gradient to direct CAR-T cells toward tumor cells.

The nanoparticle compositions and CAR-T cells described herein may interact to enhance therapeutic activity against cancerous growths. In some aspects, the nanoparticle composition may enhance CAR-T cell transmigration through tumor blood vessels. The nanoparticle composition may induce expression of adhesion molecules on tumor vascular endothelial cells, including E-selectin, intercellular adhesion molecule-1 (ICAM-1), and vascular cell adhesion molecule-1 (VCAM-1). E-selectin may slow the movement of CAR-T cells over the tumor blood vessel wall through a bind and release mechanism with sialyl Lewisx (sLex) carbohydrates. ICAM-1 may bind to the lymphocyte function-associated antigen (LFA) receptor on CAR-T cells, causing firm adhesion of the CAR-T cells to endothelial cells and regulating transmigration of the CAR-T cells through tumor blood vessels. The adhesion molecules may facilitate leukocyte rolling, adhesion, and transmigration through the endothelial barrier.

In some aspects, the nanoparticle composition may home CAR-T cells and CD8+ T cells through the tumor microenvironment by establishing a chemokine gradient. The chemokine gradient may include CXCL9, CXCL10, CXCL11, or combinations thereof. The chemokine gradient may direct CAR-T cells, natural killer (NK) cells, and T cells toward cancer cells expressing cognate antigens. In some cases, the nanoparticle composition may stimulate tumor cells and stromal fibroblasts to secrete chemokines. The chemokines may be entrapped by heparan sulfate proteoglycans within the tumor microenvironment.

In some aspects, CAR-T cells and CD8+ T cells may express CXCR3-A receptors. The CXCR3-A receptors may bind to CXCL9, CXCL10, and CXCL11 and may be associated with immune cell chemotaxis and proliferation. In some cases, the chemokine gradient may guide infiltrating leukocytes toward antigen-expressing tumors.

In some aspects, the nanoparticle composition may recruit immune system cells to the tumor site. The nanoparticle composition may induce chemokines involved in the innate immune response. In some cases, the nanoparticle composition may recruit neutrophils and macrophages to the tumor site. The recruitment of immune system cells may contribute to anti-tumor responses.

In some aspects, the nanoparticle composition may induce expression of major histocompatibility complex (MHC) class I molecules or human leukocyte antigen (HLA) molecules on tumor cells. In some cases, the nanoparticle composition may induce MHC/HLA expression on immunologically cold tumors. Immunologically cold tumors may include tumors that exhibit reduced immune cell infiltration or reduced expression of molecules involved in immune recognition. The induction of MHC-I or HLA expression may promote immune recognition and cytotoxic activity against tumor cells. In some cases, the nanoparticle composition may induce MHC-I-induced activation of CD8+ T cells. The combination of the nanoparticle composition and CAR-T cells or endogenous CD4+ and CD8+ T cells may result in an MHC/HLA-driven immune response against a solid tumor.

In some aspects, the nanoparticle composition may interact with the tumor endothelium, engaging tumor necrosis factor receptor 1 (TNFR1) and interferon gamma receptor (IFNγR1/2) signaling pathways. The engagement of TNFR1 and IFNγR1/2 signaling pathways may induce E-selectin, ICAM-1, and VCAM-1 expression on tumor vascular endothelial cells. In some cases, the nanoparticle composition may reduce tumor interstitial fluid pressure and increase perfusion within the tumor microenvironment. The reduction in tumor interstitial fluid pressure and increase in perfusion may promote deeper immune infiltration into the tumor.

In some aspects, the combination of the nanoparticle composition and CAR-T cells may result in increased intratumoral CAR-T cell infiltration and cytotoxicity relative to CAR-T cell therapy alone. The nanoparticle composition may increase the number of CAR-T cells and CD8+ T cells that cross through the tumor vasculature. In some cases, the nanoparticle composition may increase the number of CAR-T cells, CD4+ T cells, and CD8+ T cells that are delivered to cancer cells. The increased delivery of immune cells to cancer cells may increase cytotoxicity against tumor cells.

In some aspects, the methods described herein may include detecting and assessing the effects of treatment with the nanoparticle composition and the CAR-T cells. Detection and assessment methods may include methods for detecting vascular disruption, methods for assessing CAR-T cell penetration, and methods for monitoring tumor response.

In some aspects, vascular disruption may be detected by measuring a loss of endothelial cell marker CD34. CD34 is a vascular endothelial cell marker, and a decrease in CD34 signal may indicate disruption of tumor vasculature. In some cases, vascular disruption may be detected by measuring endothelial cell death. Vascular disruption may be determined by quantitative immunohistochemistry for vascular endothelial cell markers such as CD31, CD34, or von Willebrand Factor. In some aspects, vascular disruption may be determined by quantitative immunohistochemistry for pericyte markers such as platelet-derived growth factor receptor beta (PDGFR-β). In some cases, vascular disruption may be determined by quantitative immunohistochemistry for basement membrane markers such as collagen or laminin.

In some aspects, vascular disruption may be confirmed using dynamic contrast-enhanced magnetic resonance imaging (DCE-MRI). DCE-MRI may be performed prior to administration of the nanoparticle composition to establish a baseline vascular leak. In some cases, DCE-MRI may be performed after administration of the nanoparticle composition to confirm disruption of tumor blood vessels. DCE-MRI with gadolinium may be performed about 24 hours prior to treatment with the nanoparticle composition and about 24 hours after treatment with the nanoparticle composition. In some aspects, DCE-MRI may be used to determine the number of nanoparticle composition treatments administered prior to administration of CAR-T cells.

In some aspects, CAR-T cell penetration may be assessed using immunohistochemistry staining for CD3. CD3 is representative of the presence of T cells, including CAR-T cells. In some cases, tumors may be fixed, sectioned, and stained with anti-CD3 antibodies to detect CAR-T cell infiltration. In some aspects, immunohistochemistry may include staining for tumor-associated antigens, such as CD19 or mesothelin, to identify the location of CAR-T cells relative to tumor cells.

In some aspects, CAR-T cell penetration may be assessed using flow cytometry. Flow cytometry may include creating a single cell suspension from tumor tissue and staining for markers associated with CAR-T cells. In some cases, flow cytometry may be used to quantify the total number of CAR-T cells present in a tumor. In some aspects, flow cytometry may include staining for endothelial cell markers such as CD31 or CD34 and counterstaining with adhesion molecule markers such as E-selectin or intercellular adhesion molecule-1 (ICAM-1).

In some aspects, tumor response may be monitored using bioluminescence. Bioluminescence may be used with luciferase-transfected cancer cells. In some cases, cancer cells may be transfected with luciferase prior to implantation, and bioluminescence imaging may be used to monitor tumor growth over time. In some aspects, luciferase-transfected CAR-T cells may be used to track CAR-T cell distribution and localization within the tumor microenvironment. In some cases, bioluminescence signals from luciferase-transfected CAR-T cells may be combined with immunohistochemistry staining of cancer cells to assess CAR-T cell penetration and co-localization with tumor cells.

In some aspects, multicolor immunohistochemistry may be used to demonstrate colocalization of vascular endothelial cell markers with adhesion molecule markers. In some cases, multicolor immunohistochemistry may include staining for CD31 or CD34 and counterstaining with E-selectin or ICAM-1. In some aspects, immunohistochemistry may include staining tumor cells with a tumor-selective biomarker, such as mesothelin or human epidermal growth factor receptor 2 (HER2), and counterstaining with ICAM-1.

In some aspects, chemokine gradients within the tumor microenvironment may be assessed using immunohistochemistry with fluorescently labeled chemokine-specific antibodies. In some cases, chemokines may be embedded within heparan sulfate proteoglycans in the tumor microenvironment, and immunohistochemistry may be used to identify chemokine distribution within the extracellular matrix. In some aspects, fluorescent signals from labeled CAR-T cells may be combined with chemokine-specific staining to assess CAR-T cell migration toward tumor cells.

### EXAMPLES

Mice with TNFα insensitive tumor lines were subjected to a series of treatments, including administration of a nanoparticle composition and administration of CAR-T cells. The nanoparticle composition, ZB-TNFα, included colloidal gold nanoparticles conjugated to TNFα and PEG-thiol. The mice that received administration of the nanoparticle composition and the CAR-T cells were compared to mice that did not receive the nanoparticle composition and mice that did not receive the CAR-T cell administration. The studies show that when the nanoparticle composition is administered with CAR-T cells, the nanoparticle composition can increase T-cell uptake and/or reduce tumor volume compared to other treatment groups.

### Example 1

NOD scid gamma (NSG) mice (i.e., NOD.Cg-PrkdcscidII2rgtm1wjl/SzJ mice) received subcutaneous inoculations of the AsPC-1 pancreatic tumor line on Day 0. On Day 21, inoculated mice with a 200-300 mm³ tumor volume were randomized into the eight treatment groups shown in Table 1, such that each treatment group included 8 mice.

TABLE 1

| Treatment Group | ZB-TNFα Amount | CAR-T Cell Amount |
|---|---|---|
| Group 1 | 2.5 μg | 1 million cells |
| Group 2 | 5.0 μg | 1 million cells |
| Group 3 | 5.0 μg | 0.5 million cells |
| Group 4 | 5.0 μg | 0.25 million cells |
| Group 5 | Control Buffer | 1 million cells |
| Group 6 | Control Buffer | 0.5 million cells |
| Group 7 | Control Buffer | 0.25 million cells |
| Group 8 | Control Buffer | Control Media |

For each treatment group, the ZB-TNFα or control buffer was intravenously administered on Day 21, and the CAR-T cells or control media were intravenously administered one hour after the administration of ZB-TNFα. The control buffer was an aqueous solution comprising 4 mM sodium phosphate, 1 wt. % PEG-1450, and 0.1 wt. % human serum albumin (HSA). The control media comprised 100 μL phosphate buffered saline with 5 wt. % fetal bovine serum (PBS/5% FBS). The ZB-TNFα comprised the ZB-TNFα nanoparticle composition in 90 μL of the control buffer. The CAR-T cells comprised MSLN-2-TM-41BB-CD3ζ CAR-T cells suspended in 100 μL of the control media.

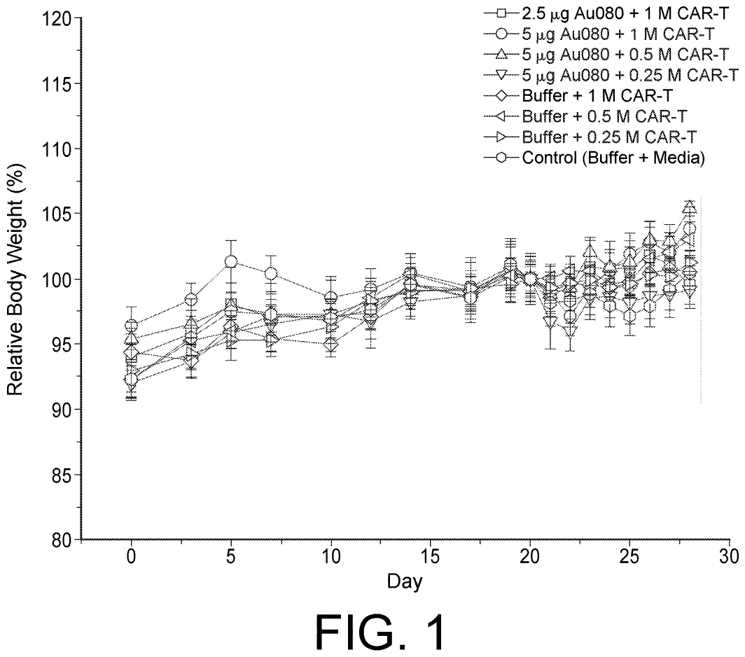

The mean relative body weight (i.e., current weight as a percentage of Day 0 weight) of each group was measured from Day 0 to Day 28. The recorded mean relative body weights for each treatment group are plotted in FIG. 1. If any of the subjects had shown a relative body weight loss of 18%, they would have been sacrificed. None of the subjects showed a weight loss of 18%, indicating a tolerability to the ZB-TNFα nanoparticle composition.

### Example 2

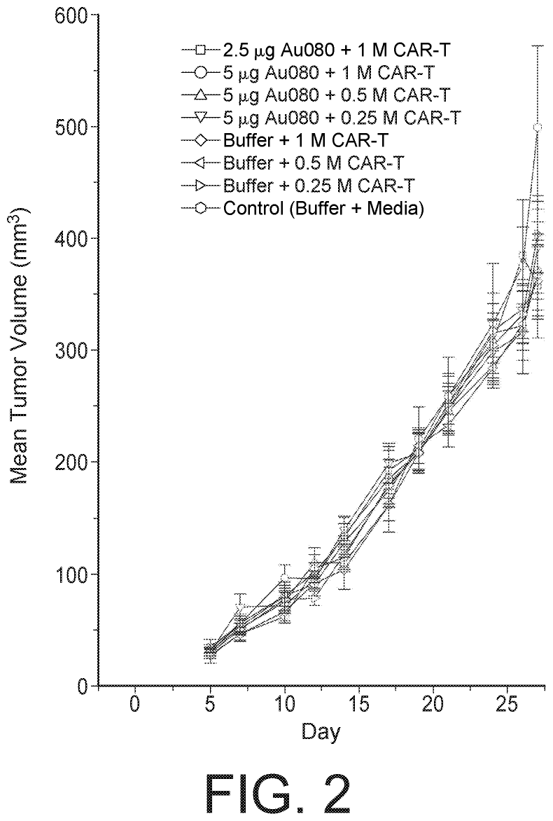

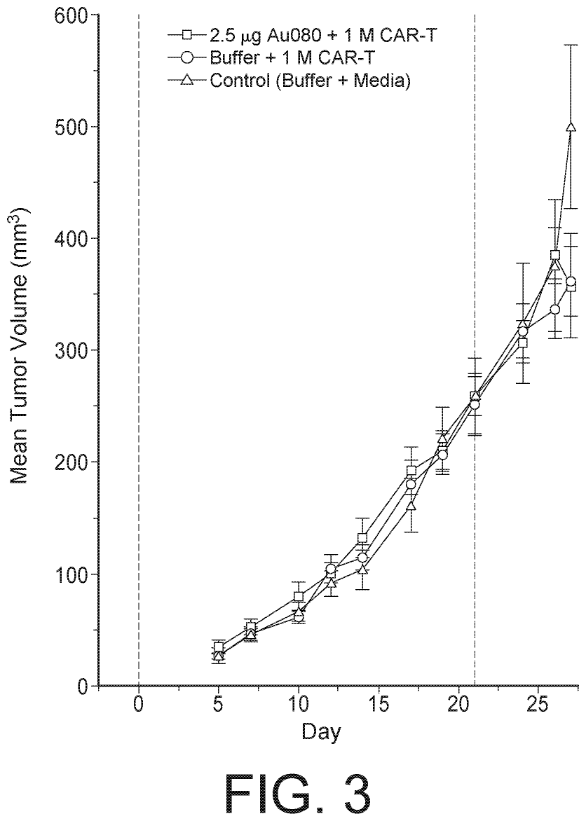

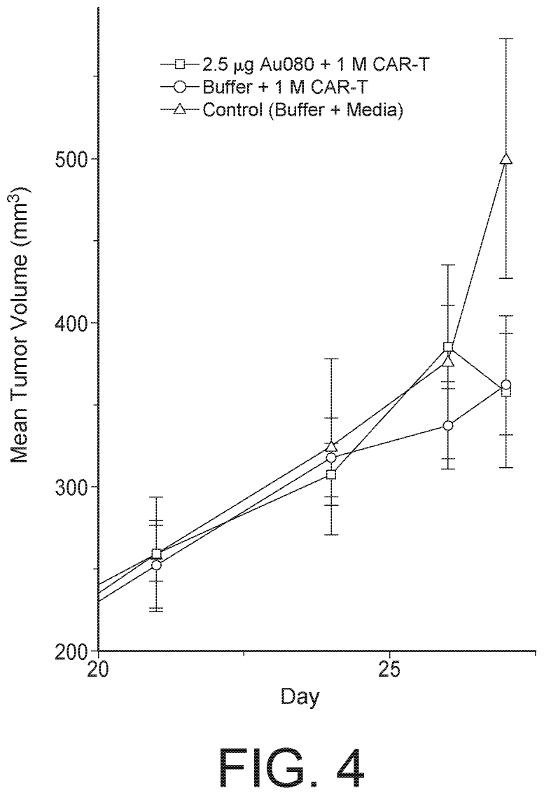

The mean tumor volume of each treatment group of the mice from Example 1 were measured from day 0 to Day 28. On Day 28, the mice were necropsied and the tumors were excised. The excised tumors were weighted. The recorded mean tumor volumes for each treatment group are plotted in FIG. 2. For clarity, the plots of only three of the treatment groups are shown in FIG. 3, and an enlargement of Days 20-28 of those treatment groups is shown in FIG. 4.

As shown in FIGS. 2-4, by Day 28, the only treatment group that showed a decrease in tumor volume was Group 1. This decrease in tumor volume was not exhibited by any of the treatment groups that did not receive ZB-TNFα, nor by the treatment groups with large doses of TNFα.

### Example 3

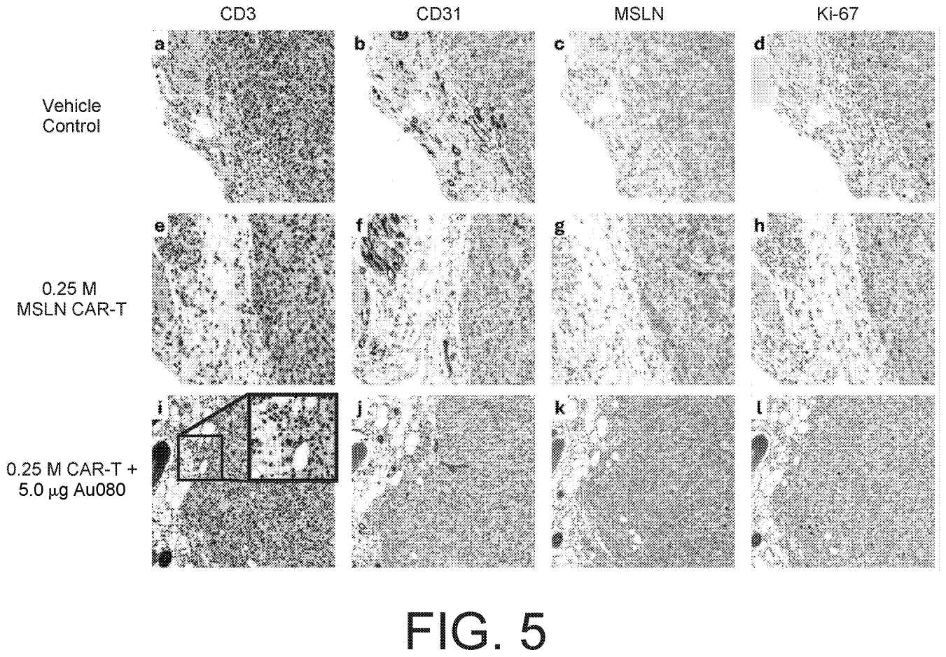

After Day 28, the excised tumors from Example 2 were sectioned. A section of each tumor was placed in formalin and processed for formalin-fixed paraffin-embedded (FFPE) for immunohistochemistry (IHC). Representative images from the RIC analysis are shown in FIG. 5.

Referring to FIG. 5, the top row [panels (a), (b), (c), (d)] are images of a tumor excised from a Group 8 mouse. The images in the top row are of different cross sections of the same tumor. The middle row [panels (e), (f), (g), (h)] are images of a tumor excised from a Group 7 mouse. The images in the middle row are of different cross sections of the same tumor. The images in the bottom row [panels (i), (j), (k), (l)] are images of a tumor excised from a Group 4 mouse. The images in the bottom row are of different cross sections of the same tumor. All images were stained blue (appearing as black dots in FIG. 5) with hematoxylin.

The images in the first column [panels (a), (e), (i)] were stained brown (appearing as grey shading in FIG. 5) with anti-CD3 antibodies. The presence of CD3 is representative of the presence of T-cells, such as, for example, CAR-T cells. The images in the second column [panels (b), (f), (j)] were stained brown (appearing as grey shading in FIG. 5) with anti-CD31 antibodies. The presence of CD31 is representative of the presence of endothelial cells. The images in the third column [panels (c), (g), (k)] were stained brown (appearing as grey shading in FIG. 5) with anti-mesothelin antibodies. The presence of mesothelin is representative of cells expressing mesothelin. The images in the fourth column [panels (d), (h), (1)] were stained brown (appearing as grey shading in FIG. 5) with anti-Ki-67 antibodies. The presence of Ki-67 is representative of proliferating cell growth.

Still referring to FIG. 5, panel (i) includes an enlargement of a region of the tumor. The grey discoloration representative of detected CD3 shows that tumor cells have uptaken T-cells (e.g., CAR-T cells). Similar degrees of uptake were not observed in the treatment groups that did not include ZB-TNFα.

### Example 4

NSG mice (i.e., NOD.Cg-PrkdcscidII2rgtm1wjl/SzJ mice) received subcutaneous inoculations of the SKOV3 ovarian tumor line on Day 0. On Day 31, inoculated mice with a 200-300 mm³ tumor volume were randomized into the eight treatment groups shown in Table 2, such that each treatment group included 8 mice.

TABLE 2

| Treatment | ZB-TNFα | ZB-TNFα | CAR-T Cell | CAR-T Cell |
|---|---|---|---|---|
| Group | Amount | Administration | Amount | Administration |
| Group 1 | 2.5 μg | Day 32 | 3 million cells | 1 hr after ZB-TNFα |
| Group 2 | 5.0 μg | Day 32 | 3 million cells | 1 hr after ZB-TNFα |
| Group 3 | 2.5 μg | Day 37 | 3 million cells | Day 32 |
| Group 4 | 1.0 μg | Day 32 | 3 million cells | 1 hr after ZB-TNFα |
| Group 5 | Control Buffer | Day 32 | 3 million cells | 1 hr after ZB-TNFα |
| Group 6 | 2.5 μg | Day 32 | Control Media | 1 hr after ZB-TNFα |
| Group 7 | Control Buffer | Day 37 | 3 million cells | Day 32 |
| Group 8 | Control Buffer | Day 32 | Control Media | 1 hr after ZB-TNFα |

For each treatment Groups 1, 2, 4, 5, 6, and 8, the ZB-TNFα or control buffer was intravenously administered on Day 32, and the CAR-T cells or control media were intravenously administered one hour after the administration of ZB-TNFα. For Groups 3 and 7, the CAR-T cells were administered on Day 32, and the ZB-TNFα or control buffer was administered on Day 37. The control buffer was an aqueous solution comprising 4 mM sodium phosphate, 1 wt. % PEG-1450, and 0.1 wt. % human serum albumin (HSA). The control media comprised 100 μL phosphate buffered saline with 5 wt. % fetal bovine serum (PBS/5% FBS). The ZB-TNFα comprised the ZB-TNFα nanoparticle composition in 90 μL of the control buffer. The CAR-T cells comprised HER2-TM-41BB-CD3ζ CAR-T cells suspended in 100 μL of the control media.

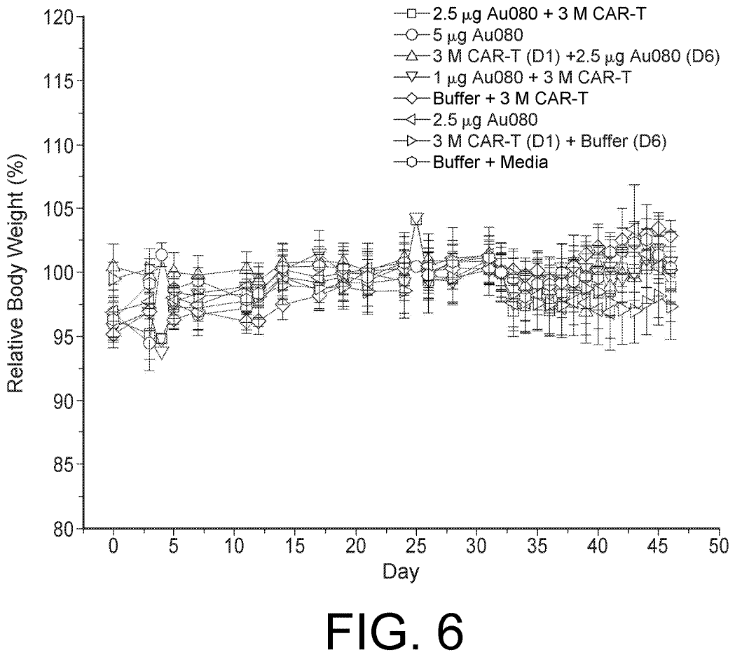

The mean relative body weight (i.e., current weight as a percentage of Day 0 weight) of each group was measured from Day 0 to Day 41. Due to low tolerability (i.e., relative body mass dropping by more than 18%), Group 2 was sacrificed on Day 32. Due to hindlimb weakness (an unrelated condition common in NSG mice), one mouse in Group 1, one mouse in Group 5, two mice in Group 6, and one mouse in Group 8, were sacrificed early (i.e., Day 40 or Day 41). The recorded mean relative body weights for each treatment group are plotted in FIG. 6.

### Example 5

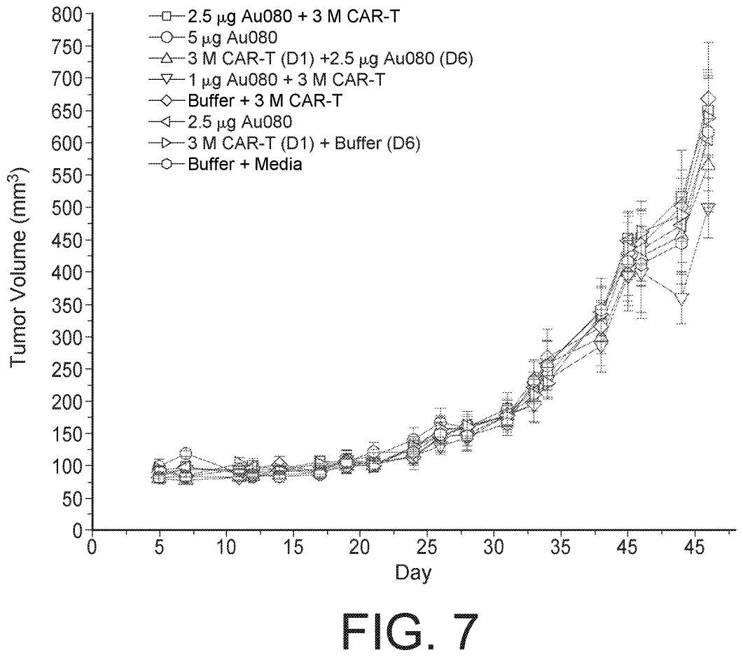

The mean tumor volume of the mice from Example 4 were measured from day 0 to Day 47. The recorded mean tumor volumes for each treatment group are plotted in FIG. 7.

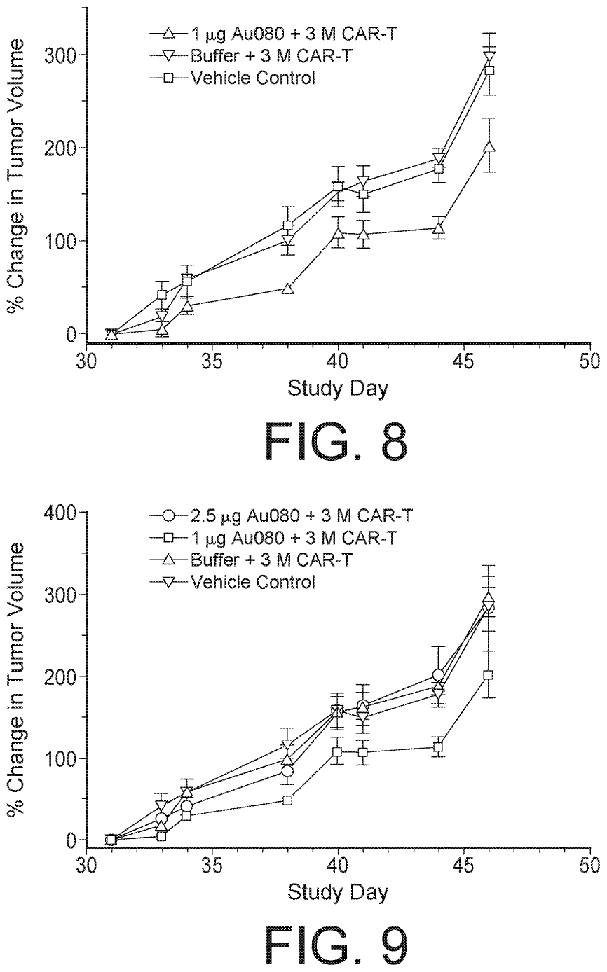

FIG. 8 shows the tumor volumes of Group 4 (upward-pointing triangles), Group 5 (downward-pointing triangles), and Group 8 (squares), represented as percent change in tumor volume. Group 4 showed a statistically significant (p<0.05) lower change in tumor volume compared to groups that did not receive ZB-TNFα, as determined by t-Test (n=7).

FIG. 9 shows the tumor volumes of Group 1 (circles), Group 4 (squares), Group 5 (upward-pointing triangles), and Group 8 (downward-pointing triangles) represented as percent change in tumor volume. Group 4 had a statistically significant (p<0.05) lower change in tumor volume compared to groups that did not receive ZB-TNFα, as determined by t-Test (n=7). Statistical significance between Group 4 and Group 1 was found at a p value less than 0.11.

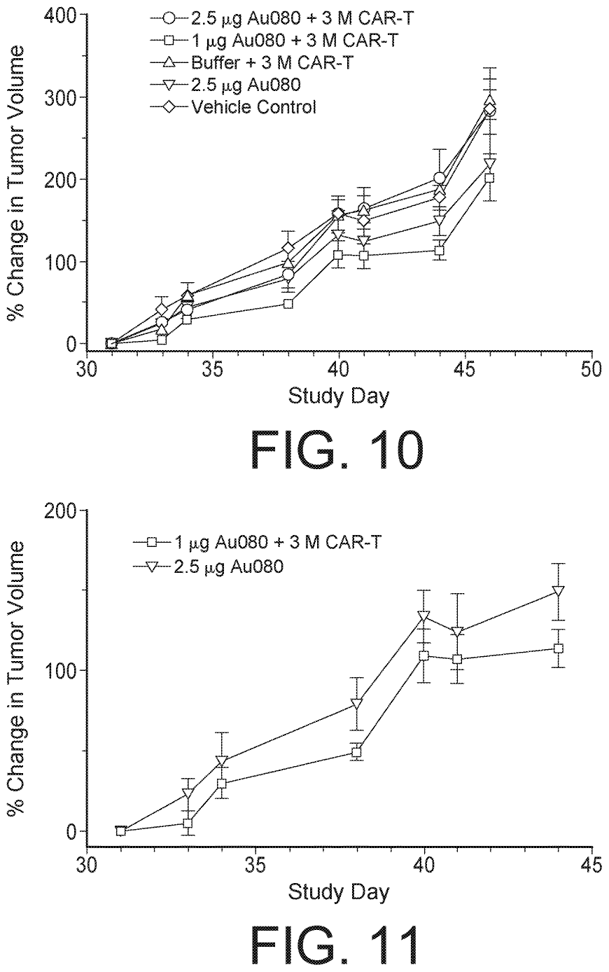

FIG. 10 shows the tumor volumes of Group 1 (circles), Group 4 (squares), Group 5 (upward-pointing triangles), Group 6 (downward-pointing triangles), and Group 8 (diamonds) represented as percent change in tumor volume. Group 4 had a statistically significant (p<0.05) lower change in tumor volume compared to groups that did not receive ZB-TNFα, as determined by t-Test (n=7). Statistical significance between Group 4 and Group 6 was found at a p value less than 0.33.

FIG. 11 shows the tumor volumes of Group 4 (squares) and Group 6 (downward-pointing triangles), represented as percent change in tumor volume. At day 44, Group 4 showed statistically significant (p<0.1) lower change in tumor volume compared to Group 6, as determined by t-Test (n=6).

### Example 6

Tumor samples of the mice sacrificed early on Days 40 and 41 due to hind-limb weakness from Example 4 were analyzed via flow cytometry. The flow cytometry protocol gated beads, forward scatter versus side scatter, and single cells, and live cells to determine the number of live cells in the sample. The live cells were gated into (i) hcD45+ (representing human CD-45-positive leukocytes); (ii) hCD45−mCD45− (representing non-human, non-mouse-CD45-positive cells); and (iii) mCD45+ (mouse CD45-positive leukocytes). The hcD45+ cells were gated into CD3+ cells, and the CD3+ cells were further gated to CAR+ cells. The hCD45−mCD45− cells were gated into Her2+ cells.

The flow cytometry data for tumor samples of the mice sacrificed early from Example 4 are shown in Tables 3-6. Table 3 corresponds to the tumor sample from the Group 1 mouse, Table 4 corresponds to the tumor sample from the Group 5 mouse, Table 5 corresponds to the tumor sample from the Group 6 mouse, and Table 6 corresponds to the tumor sample from the Group 8 mouse.

TABLE 3

| Gate | | Count | Percent Gated |
|---|---|---|---|
| All | | 722,672 | 100.00 |
| Beads | | 38,945 | 5.39 |
| FSC vs. SSC | | 480,007 | 66.42 |
| Single Cells | | 473,533 | 98.65 |
| Live Cells | | 237,773 | 50.21 |
| hcD45+ | | 316 | 0.13 |
| hcD45+ | CD3+ | 109 | 34.49 |

| | | | | |
|---|---|---|---|---|
| hcD45+ | CD3+ | CAR+ | 8 | 7.34 |

| hCD45−mCD45− | | 147,966 | 62.23 |
|---|---|---|---|
| hCD45−mCD45− | Her2+ | 126,128 | 85.24 |

| | | |
|---|---|---|
| mCD45+ | 89,486 | 37.64 |

TABLE 4

| Gate | | Count | Percent Gated |
|---|---|---|---|
| All | | 27,959 | 100.00 |
| Beads | | 2,199 | 7.87 |
| FSC vs. SSC | | 16,018 | 57.29 |
| Single Cells | | 15,790 | 98.58 |
| Live Cells | | 9,817 | 62.17 |
| hcD45+ | | 15 | 0.15 |
| hcD45+ | CD3+ | 7 | 46.67 |

| | | | | |
|---|---|---|---|---|
| hcD45+ | CD3+ | CAR+ | 1 | 14.29 |

| hCD45−mCD45− | | 7,150 | 72.83 |
|---|---|---|---|
| hCD45−mCD45− | Her2+ | 5,777 | 80.80 |

| | | |
|---|---|---|
| mCD45+ | 2,652 | 27.01 |

TABLE 5

| Gate | | Count | Percent Gated |
|---|---|---|---|
| All | | 1,018,625 | 100.00 |
| Beads | | 36,329 | 3.57 |
| FSC vs. SSC | | 701,333 | 68.85 |
| Single Cells | | 693,027 | 98.82 |
| Live Cells | | 372,035 | 53.68 |
| hcD45+ | | 456 | 0.12 |
| hcD45+ | CD3+ | 0 | 0.00 |

| | | | | |
|---|---|---|---|---|
| hcD45+ | CD3+ | CAR+ | 0 | 0.00 |

| hCD45−mCD45− | | 196,300 | 52.76 |
|---|---|---|---|
| hCD45−mCD45− | Her2+ | 169,501 | 86.35 |

| | | |
|---|---|---|
| mCD45+ | 175,268 | 47.11 |

TABLE 6

| Gate | | Count | Percent Gated |
|---|---|---|---|
| All | | 44,008 | 100.00 |
| Beads | | 2,065 | 4.69 |
| FSC vs. SSC | | 27,362 | 62.18 |
| Single Cells | | 26,964 | 98.55 |
| Live Cells | | 13,807 | 51.21 |
| hcD45+ | | 8 | 0.06 |
| hcD45+ | CD3+ | 1 | 12.50 |

| | | | | |
|---|---|---|---|---|
| hcD45+ | CD3+ | CAR+ | 1 | 100.00 |

| hCD45−mCD45− | | 10,132 | 73.38 |
|---|---|---|---|
| hCD45−mCD45− | Her2+ | 7,730 | 76.29 |

| | | |
|---|---|---|
| mCD45+ | 3,666 | 26.55 |

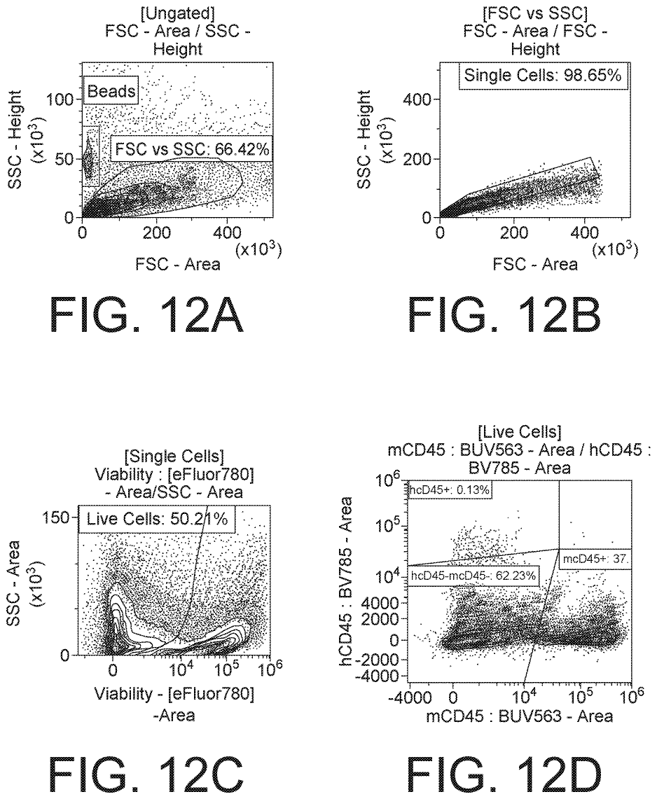

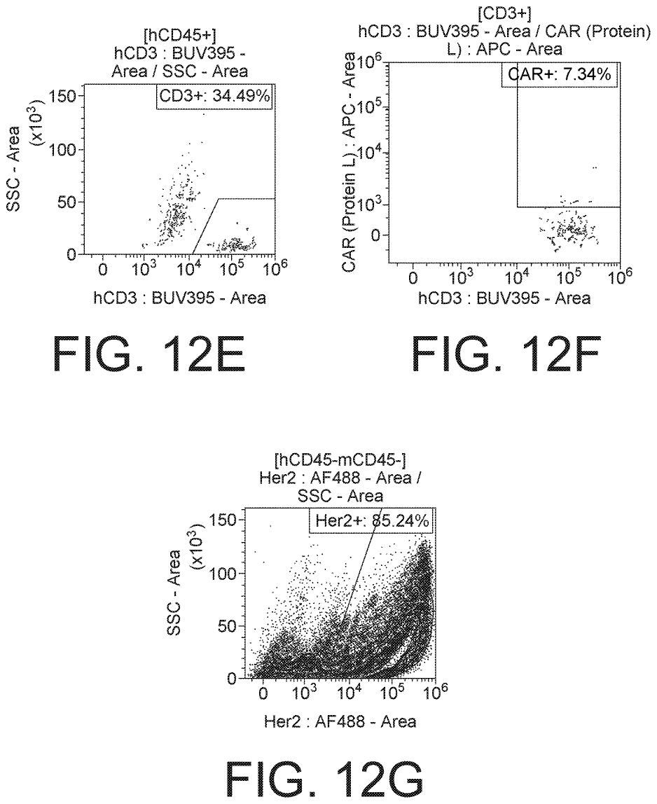

The flow cytometry plots from the analysis of the tumor sample of Group 1 are shown in FIGS. 12A-G. The flow cytometry data from Group 1 contained a detectable human CD45+CD3 population and a smaller CAR-positive subset within those T cells. This demonstrates low-level CAR-T presence in tumor tissue. Tumor samples from the animals in CAR-T only, ZB-TNFα only, and vehicle control (control buffer and control media only) did not show T-cell or CAR-T uptake above the limit of quantification.

Further additional studies are planned to evaluate additional tumor models and administration methods.

### Study 1: Induction of Vascular Disruption of MCL Tumors by a Single Injection of ZB-TNFα

Briefly, 2×10⁶ luciferase transfected JeKo-1 cancer cells will be implanted, via an intravenous injection, into NOD-SCID-γ chain−/− (NSG) mice. At day 7, the mice will be randomized into groups with n=3 and treated with saline, PEGylated gold nanoparticles at the gold equivalence of 10 μg of ZB-TNFα, ZB-TNFα at 5.0, 7.5 and 10 μg of TNF, administered as ZB-TNFα. Twenty-four hours after ZB-TNFα treatment, the tumors will be harvested and fixed.

Vascular disruption will be determined by quantitative immunohistochemistry for the following markers: CD31 (vascular endothelial cell marker), CD34 (vascular endothelial cell marker), von Willebrand Factor (vascular endothelial cell marker), PDGFR-b (pericyte marker), collagen (basement membrane marker), laminin (basement membrane markers). Other markers may also be used. The sections will be scored by a blinded, board-certified pathologist using the general scheme of ++++ for no effect, +++ slight decrease in the IHC signal, ++ significant decrease in IHC signal, + to 0+ for no detectable signal. Statistical difference will be determined by a Mann-Whitney U Test.

### Study 2: Induction of Vascular Disruption of MCL Tumors by Multiple Injections of ZB-TNFα

This study will includes aspects of the methods of Study 1. In this study, the number of treatments will be increased from 1 to 2 or 3 treatments with ZB-TNFα, and dynamic contrast-enhance magnetic resonance imaging (DCE-MRI) will be used to confirm vascular disruption.

After implantation, MCL-tumored mice will receive 1, 2, or 3 injections of 7.5 μg of ZB-TNFα (n=3 treatment group). The number of treatments may be altered pending the data obtained in Study 1. Treatments include saline, ZB-TNFα, PEGylated gold controls (administered at the gold equivalence as the 7.5 μg dose of ZB-TNFα, and the excipients used in formulating ZB-TNFα.

DCE-MRI with gadolinium will be performed 24 hours prior to treatment with ZB-TNFα as well as 24 hours after each round of treatments with ZB-TNFα. The MRI scanning will include aspects of the protocols outlined by Nilubol et al., “Novel dual-action targeted nanomedicine in mice with metastatic thyroid cancer and pancreatic neuroendocrine tumors”, J Natl Cancer Inst., 2018, volume 110, issue 9, pages 1019-1029. The MRI scanning will identify the number of ZB-TNFα treatments needed to maximally disrupt the tumor vasculature prior to the administration of CAR-T cells.

Study 3: Impact of ZB-TNFα on Penetration of CAR-T Cells into MCL Tumors

The data from Study 1 and Study 2 will help in developing a treatment protocol to evaluate the impact of ZB-TNFα on the penetration of CAR-T cells into MCL tumors. For illustration purposes, the study described below is based on an expected result that one treatment with 7.5 μg of ZB-TNFα maximally disrupts the tumor vasculature.

Study 3 is intended to demonstrate that vascular disruption induced by compositions of the present disclosure result in an increase in the number of CAR-T cell that penetrate the tumor. For clarity, this study is summarized in Table 7, below.

TABLE 7
Study Design to Evaluate Enhancement of CAR-T Cell Penetration into MCLs

| Day of | | | |
|---|---|---|---|
| experiment | Treatment | Output | Comments |
| 0 | Implantation of target+ and | Establish tumors | 1-Target+ (JeKo-1 |
| | target− tumor cells | | cells) and target− |
| 6 | Pre-Rxment MRI | Establish Baseline | (K562: should from |
| | | vascular leak | tumors in NSG mice) |
| 7 | Rxment with 7.5 μg ZB- | N/A | cancer cells will be |
| | TNFα | | implanted into |
| 8 | Post-Rxment MRI | Confirm maximal | sufficient number of |
| | | disruption of tumor | animals to |
| | | blood vessels | accommodate a |
| 8 | Injection of 0.75⁶ Target+ | N/A | minimum of an n = 3-5/ |
| | and target− CAR-T-cells | | drug and controls |
| 9 or later | Sacrifice animals | Prepare tumor for | 2-Drugs: 7.5 μg ZB- |
| | IHC and Flow analysis to | Flow and ICH | TNFα, PEGylated gold |
| | detect CAR-T cell penetration | analysis | equivalence, excipients. |
| TBD | | Analyze tumors for | The drugs will be |
| | | the total number of | administered alone or in |
| | | CAR-T cells present | combination with CAR- |
| | | in tumor | T therapies. |

CD19 positive or CD19 negative cancer cells will be implanted on day 0 of the study. On day 6 after implantation, a pre-treatment MRI will be conducted to establish a baseline vascular leak. On day 7 the animals will receive an IV injection of 7.5 μg of ZB-TNFα or control drugs. On day 8, a second MRI scan will be performed to confirm vascular disruption. On the same day, the animals will receive an IV injection of luciferase transfected target positive or target negative CAR-T cells.

Commercial CD-19 targeting CAR-T cells will be used for these studies. These cells may be screened in vitro to confirm their cytotoxicity/potency against the target cells (JeKo-1), and the number of CAR-T cells may be adjusted. As a starting point, 0.5×10⁶ cells may be administered. This relatively low dose of cells is that it allows for clear signals in efficacy to be generated if ZB-TNFα enhances the penetration of the CAR-T cells into the tumor.

To assess the impact of ZB-TNFα on CAR-T cell penetration, animals will be sacrificed one and two days after the CAR-T cells are administered. For IHC based studies, the tumors will be fixed, sectioned, and stained for CD19 (the putative target). Both the luciferase signal coming from the CAR-T cells along with a complimentary stain coming from the IHC CD19 staining of the cancer cells, will be imaged.

Treatment groups will include ZB-TNFα along with controls such as saline, one or more excipients, and PEGylated gold nanoparticles. Target positive and control cell lines: Target (CD19) positive (JeKo-1) and negative (K562) cell lines. For CAR-T, CD19 targeting CAR-T and/or transfection controls lacking the targeting moieties but containing the effector molecules may be used.

### Study 4: Impact of ZB-TNFα on Safety and Efficacy of CAR-T Cell Therapy for MCL Tumors

The impact of ZB-TNFα on the safety and efficacy of CAR-T cells in treating MCL tumors will be evaluated. Luciferase transfected cancer cell lines will be used to allow the use of bioluminescence to monitor tumor growth. The experimental protocol will include aspects of that described in Table 2 with the major difference being that we will administer 0.25×10⁶, 0.5×10⁶, and 1.0×10⁶ CAR-T cells. Tumor responses will be monitored using bioluminescence.

It is hypothesized that by inducing selective disruption of the tumor vasculature ZB-TNFα will enhance the penetration of CAR-T cells into MCL tumors. In doing so, ZB-TNFα may facilitate the use of lower CAR-T cell dose without compromising efficacy. In using lower number of CAR-T cells, ZB-TNFα may improve the safety of CAR-T treatment in solid tumors. For example, by using lower cell numbers the ZB-TNFα-CAR-T treatments may prevent the major dose limiting toxicities associated with CAR-T treatment, such as Cytokine Release Syndrome.

The above description and examples are illustrative, and not intended to be restrictive. For example, and as has been described, the above-described embodiments (and/or aspects thereof) may be used in combination with each other.

What is claimed is:

1. A method of treating a cancerous growth, the method comprising:
administering a nanoparticle composition to a subject having the cancerous growth; and
administering CAR-T cells to the subject.

2. The method of claim 1, wherein the CAR-T cells are administered about 15 minutes to about 8 hours after the nanoparticle composition is administered.

3. The method of claim 1, wherein the CAR-T cells are administered about 15 minutes to about 45 minutes after the nanoparticle composition is administered.

4. The method of claim 1, wherein administering the nanoparticle composition to the subject includes administering about 50 μg to about 1000 μg of the nanoparticle composition.

5. The method of claim 1, wherein administering CAR-T cells includes administering about 0.1×10⁶ to about 10×10⁹ CAR-T cells.

6. The method of claim 1, wherein the nanoparticle composition comprises a colloidal gold nanoparticle.

7. The method of claim 1, wherein the nanoparticle composition comprises tumor necrosis factor alpha.

8. The method of claim 1, wherein the nanoparticle composition comprises interferon gamma, interleukin-12, or a combination thereof.

9. The method of claim 1, wherein the cancerous growth includes a solid tumor.

10. The method of claim 1, further comprising measuring an interstitial fluid pressure of the solid tumor.

11. The method of claim 1, further comprising determining a dosage of the CAR-T cells based on the measured interstitial fluid pressure.

12. The method of claim 1, further comprising determining a dosage of the nanoparticle composition based on the measured interstitial fluid pressure.

13. The method of claim 1, further comprising determining a number of administrations of the nanoparticle composition based on the measured interstitial fluid pressure.

14. The method of claim 1, wherein the cancerous growth includes a solid tumor with an interstitial fluid pressure of at least 2.5 mmHg.

15. A method of treating a cancerous growth, the method comprising:
administering a first nanoparticle composition to a subject having the cancerous growth; and
after a circulation period, administering CAR-T cells to the subject, wherein the circulation period about 30 minutes to 8 hours after the first nanoparticle composition is administered; and
after administering CAR-T cells to the subject, administering a second nanoparticle composition to the subject.

16. The method of claim 15, wherein the cancerous growth includes a tumor, and the method further comprises:
determining an interstitial fluid pressure of the tumor; and
determining the circulation period based on the interstitial fluid pressure of the tumor.

17. The method of claim 15, wherein the cancerous growth includes a pancreatic cancer tumor or an ovarian cancer tumor.

18. The method of claim 15, wherein the cancerous growth includes a pancreatic cancer tumor and the CAR-T cells comprise MSLN-2-TM-41BB-CD3ζ CAR-T cells.

19. The method of claim 15, wherein the cancerous growth includes an ovarian cancer tumor and the CAR-T cells comprise HER2-TM-41BB-CD3ζ.

20. The method of claim 15, wherein:
the first nanoparticle composition comprises a first molar ratio of tumor necrosis factor alpha to interferon gamma and the second nanoparticle composition comprises a second molar ratio of tumor necrosis factor alpha to interferon gamma; and
the first molar ratio is greater than the second molar ratio; or
the second molar ratio is greater than the first molar ratio.
